(19) United States
(12) Patent Application Publication
(10) Pub. No.: US 2023/0393152 A1
(43) Pub. Date: Dec. 7, 2023

(54) METABOLIC MARKERS SPECIFIC TO NEURODEGENERATIVE DISEASES SUCH AS PARKINSON'S DISEASE AND USE THEREOF IN DIAGNOSIS

(71) Applicants: UNIVERSITE GRENOBLE ALPES, Saint Martin D'Heres (FR); INSERM - Institut National de la Santé et de la Recherche Médicale, Paris (FR)

(72) Inventors: Sabrina BOULET, Voreppe (FR); Florence FAUVELLE, Grenoble (FR); David MALLET, Le Fontanil-Cornillon (FR)

(73) Assignees: UNIVERSITE GRENOBLE ALPES, Saint Martin D'Heres (FR); INSERM - Institut National de la Santé et de la Recherche Médicale, Paris (FR)

(21) Appl. No.: 18/032,384

(22) PCT Filed: Nov. 18, 2021
(86) PCT No.: PCT/FR2021/052035

§ 371 Date: Apr. 18, 2023

(30) Foreign Application Priority Data
Nov. 18, 2020 (FR) FR2011817

(51) Int. Cl. G01N 33/68

(52) U.S. Cl. CPC G01N 33/6896; G01N 2800/2835; G01N 2800/7066

(57) ABSTRACT

An identification of novel combinations of specific metabolites for neurodegenerative diseases for use as biomarkers in the early diagnosis of neurodegenerative diseases.

The invention concerns the identification of novel combinations of metabolites specific for neurodegenerative diseases, in particular Parkinson's disease (PD), for a use as biomarkers in the early diagnosis of neurodegenerative diseases, in particular Parkinson's disease at prodromal or de novo stage.

In the description below, references in square brackets ([ ]) refer to the list of references at the end of the text.

### STATE OF THE ART

Parkinson's disease (PD) is one of the most common neurodegenerative diseases worldwide, affecting approximately 1% of adults over the age of 60 [1]. As soon as it appears, the pathology evolves gradually and continuously according to three stages, i.e. preclinical, prodromal, and clinical stage, corresponding respectively to the appearance of neurodegeneration without symptoms (preclinical phase), then the progression of the neurodegenerative process leads to the appearance of non-motor symptoms (prodromal phase), and finally to the appearance of motor symptoms (clinical phase) [2]. It is these symptoms, present only in the last stage, which are sufficiently characteristic of PD to allow its diagnosis [3]. However, they appear when 70-80% of the dopaminergic neurons of the nigrostriatal pathway have already degenerated [1], therefore generally too late to consider neuroprotective intervention [4]. Furthermore, post-mortem histopathological analyzes of brains show that currently the diagnostic accuracy of PD in patients with motor symptoms for less than five years [5] is only 53%. Thus, too late diagnosis and its inaccuracy [3, 6, 7] strongly contribute to limited and only symptomatic patient management and therapies which, moreover, become ineffective after several years.

Moreover, although many molecules have already been tested and have shown promising theoretical or preclinical evidence of efficacy, there is still no agent or neuroprotective therapy allowing slowing down, stopping or effectively reversing neurodegeneration in patients with PD, in part because these therapeutic strategies are applied too late [8-10].

Thus, being able to detect PD with a high degree of specificity, during the early phases, especially in patients in the prodromal phase or newly and recently diagnosed (de novo early clinical phase), represents a crucial challenge to enable the success of curative or preventive strategies of PD, and the development of new therapies capable of delaying or slowing down its progression [11, 12].

The «prodromal» phase is characterized by a set of non-motor symptoms including neuropsychiatric disorders (e.g. apathy, depression, anxiety), Although they are expressed in 40 to 50% of Parkinson's patients, they are not exclusive to the disease and can be encountered in other pathologies. Patients newly and recently diagnosed with Parkinson's disease correspond to an early clinical phase called «de novo» where neuroprotective intervention can still be considered to slow the progression of the disease.

Thus, finding reliable and specific biomarkers of the prodromal or de novo phase, suitable and highly predictive for a clinical use, is an important challenge, which motivated the study of the inventors. Their starting hypothesis is that the metabolome—i.e. the global pool of metabolites—which results from the expression of genome but also reflects the impact of many environmental factors [15], could contain potential biomarkers of the disease [16-19], now considered as a multifactorial disease resulting from complex interactions between genetic and environmental factors [14]. Moreover, metabolic alterations have already been described in patients expressing neuropsychiatric symptoms similar to those observed in the prodromal phase of PD [20]. In addition, it has been shown that the analysis of metabolic deregulations observed in the serum of patients newly diagnosed with PD made it possible to precisely distinguish them from healthy subjects [21]. Finally, alterations in the plasma metabolome have been correlated with disease progression in Parkinsonian patients [22]. However, the study of the metabolome as a tool for predicting the prodromal phase of PD has never been carried out. To access this prodromal phase, the use of animal models is essential. Nevertheless, although many animal models of PD have been developed, none recapitulates all the phenotypic and etiological characteristics of the disease [23].

Thus, although the early detection of PD represents a key step for the development of curative therapies or capable of delaying or slowing down its progression, there is currently no specific tool to allow the diagnosis of patients with PD before the appearance of motor symptoms, in particular during the prodromal phase. The discovery of easily measurable and highly predictive biomarkers of the prodromal phase of the disease appears as a milestone for accelerating the development of curative therapies and improving patient care.

### DESCRIPTION OF THE INVENTION

Also, the inventors have developed an original strategy making it possible to propose new biomarkers capable of specifically and early diagnosing a neurodegenerative disease, for example Parkinson's disease (PD), in particular Parkinson's disease at the prodromal or de nova stage. This strategy, based on the use of different animal models and different patient cohorts, overcame the intrinsic limitations and increased the incomplete predictive value of each model taken individually, revealing a characteristic set of deregulated metabolites usable as a potential biomarker. Finally, the study was extended to human serum samples from two biobanks in order to validate the clinical relevance of this new PD biomarker.

Thus, via behavioral and histological assessment and using metabolomic approaches based on nuclear magnetic resonance (NMR) spectroscopy followed by advanced multivariate statistics, the inventors studied the metabolic changes (i) in serum and brain tissue of three different animal models of PD, two rodent models and a non-human primate model, mimicking the different phases of the disease [24-26], including the prodromal phase, and (ii) in the serum of two cohorts of patients with de novo PD (n=95) (United States, Italy). Deregulations common to serum and tissue samples have been observed in animals, showing that blood might reflect central (brain) dysfunction [27], at least partially.

The 6-hydroxydopamine (6-OHDA) rat model was first used because it stably mimics each stage of PD, with no evolution between stages, and it has already been validated for the study of prodromal phases [24]. Moreover, it represents a phenotypically correct model of PD, with good isomorphism and good predictive value [28], although it is etiologically limited. The α-synuclein rat model was then used because it has the advantage of being progressive in the establishment of the phenotype, which makes longitudinal studies possible. In addition, it is closer to human pathology, especially with regard to the anatomo-pathological characteristics and the physiopathological mechanisms of the disease [30].

Finally, non-human MPTP (1-methyl-4-phenyl-1,2,3,6-tetrahydropyridine) primates were used because they share greater genome sequence identity with humans and important neuroanatomical similarities.

Metabolic changes were analyzed at the blood (serum) level in order to find metabolites that are easy to be transposed into the clinic, and in specific regions of the brain differently affected by the neurodegenerative process (the dorsal striatum (DS) and the nucleus accumbens (NAcc)) in order to potentially associate changes in these metabolites to the pathophysiological processes involved in PD.

From a translational perspective, the metabolomic study was extended to serums from newly diagnosed parkinsonian patients (de novo phase) from two different biobanks (United States (NIH)+/−2 patients, Italy). The translational strategy revealed common metabolic deregulations in the serum of different animal models and patients with PD. Some of them have been found in tissue samples (animal models), possibly reflecting pathophysiological mechanisms associated with development of the PD.

Thus, this inter-species study has made it possible to reinforce the relevance of circulating metabolites for the diagnosis of PD. In addition, it proposes a composite biomarker constructed with a combination of 6 metabolites to distinguish animals mimicking PD from control animals (sham), regardless of the stage of the disease, and in particular from the prodromal or de novo phase. It is important to note that the combination of the same metabolites made it possible to distinguish patients with PD from healthy subjects.

It is interesting to note that in animals, certain metabolic dysregulations appeared before the motor symptoms and could thus represent early biomarkers of the disease.

Thus, the inventors have demonstrated that the relative concentrations of 6 metabolites: acetoacetate, betaine, β-hydroxybutyrate (BHB), creatine, pyruvate, and valine, combined together according to a logistic regression method, could constitute a precise composite biomarker of PD, in animals and in humans.

The use of this biomarker made it possible to distinguish American parkinsonian patients from healthy controls (comparison group) with a high sensitivity (0.95), and Italian parkinsonian patients with a slightly lower sensitivity (0.83). The divergent pre-analytical procedure applied for serum collection in the two cohorts (that is to say used tubes, sample processing, etc.) could be a major source of experimental variability that could explain this difference [31, 32]. Furthermore, it should be noted that the human samples were collected according to a very strict and standardized experimental protocol, but which was not designed for metabolomics. This reflects the power of metabolomics and the robustness of the results, and supports the view that serum biomarkers, easier to use and less invasive than imaging methods, could show similar efficacy [33].

Interestingly, of the six metabolites used to construct the composite PD biomarker in animal or human models, betaine, BHB, and pyruvate dysregulation were corrected by chronic treatment with an agonist dopaminergic (i.e. Pramipexole or Pra), which corrects also motivational deficits in 6-OHDA rats mimicking the prodromal phase. This pharmacological validation highlights their potential involvement in cellular dysregulations involved in neuropsychiatric symptoms associated with the prodromal or de novo stage. The metabolic deregulations reported in the present study could therefore also provide some clues about the mechanisms involved in the progression of PD.

In summary, the present study reported, for the first time, common serum metabolic alterations in three animal models mimicking PD, although from different cohorts and species. Consequently, this common signature is likely to reflect specific alterations related to the pathophysiology of PD. In particular, specific deregulations characteristic of the prodromal or de novo stage have been highlighted. In addition to providing clues for further study of metabolic alterations in animal models, metabolic dysregulations have also been found in the serums of newly diagnosed Parkinsonian patients (that is to say in the de novo clinical phase) from two biobanks, not specifically designed for metabolomics. Logistic regression, using the levels of the same metabolites used for animals, allowed a very adequate discrimination between healthy subjects and patients with PD in two cohorts of different origins (at least 0.83).

Overall, the present cross-species study demonstrated the utility and reproducibility of untargeted metabolomics as a non-invasive approach for PD biomarker research. This approach seems particularly promising for patients with PD, as the serum is easily accessible, and the biomarker may be relevant even in the early stages of disease when other methods fail.

The subject of the present invention is therefore an in vitro method for the early diagnosis of a neurodegenerative disease from a biological sample of a patient comprising:
a) the in vitro measurement in said biological sample of the expression levels (E) of at least the following 6 metabolites: acetoacetate, betaine, β-hydroxybutyrate (BHB), creatine, pyruvate, and valine;
b) the comparison of the expression levels of the metabolites measured in step a) with those of a reference biological sample or with reference values;
where a difference (e.g. joint dysregulation) of the expression levels of the metabolites measured in step a) compared to those of the reference biological sample or to reference values is the indication that said patient is suffering from the neurodegenerative disease.

According to a particular mode of the method of the present invention, step a) further comprises the in vitro measurement of the level of expression of at least one metabolite selected from the group consisting of alanine, lactate, DMSO2, glycine, serine, threonine, myo-inositol, and leucine.

According to a particular mode of the method of the present invention, step a) comprises or consists of measuring the levels of expression of acetoacetate, betaine, β-hydroxybutyrate (BHB), creatine, pyruvate, and the valine.

The term «early diagnosis», within the meaning of the present invention, means a diagnosis made at a stage of the neurodegenerative disease where the discriminating symptoms have not yet appeared or in newly and recently diagnosed patients. For example, a diagnosis would be considered early if performed at the prodromal or de novo stage of PD.

The term «neurodegenerative disease» within the meaning of the present invention means a progressive pathology which affects the brain or more generally the central nervous system, resulting in the death of nerve cells. The most famous and most frequent are Alzheimer's disease and Parkinson's disease, but there are others (multiple sclerosis, Huntington's disease, Creutzfeldt-Jakob disease, amyotrophic lateral sclerosis (also called Charcot's disease), . . . ). In particular, it is about Parkinson's disease.

The term «biological sample» within the meaning of the present invention means a biological sample (e.g. of bodily fluid (serum, blood, urine, saliva, etc.), or tissue (brain, marrow, liver, skin, etc.) taken after a 12-hour fast in a patient.

The term «biological reference sample» within the meaning of the present invention means a biological sample (e.g. of bodily fluid (serum, blood, urine, saliva), or tissue (brain, marrow, liver, skin, etc.)) taken after a 12-hour fast in a healthy individual (control/comparison group/sham), that is to say presenting no neurological disease, in particular no symptoms of PD or who may be a forerunner of PD, no family history of movement disorder and/or PD, and no specific medical conditions.

The term «reference value» within the meaning of the present invention, the value of the level of expression of a metabolite in a healthy individual, that is to say not presenting any neurological disease, in particular no symptoms of PM or may be a precursor to PD, no family history of movement disorder and/or PD, and no specific medical conditions.

The term «difference in expression levels» means, within the meaning of the present invention, the variation in the expression levels of the metabolites measured in a patient compared to the expression levels of the same metabolites in a healthy individual (reference values), that is to say having no neurological disease; in particular no symptoms of PD or which may be a precursor to PD, no family history of movement disorder and/or PD, and no specific medical condition. For example, in the case of Parkinson's disease.

The subject of the present invention is an in vitro method for the early diagnosis of Parkinson's disease from a biological sample from a patient comprising:
a) the in vitro measurement in said biological sample of the expression levels of the following metabolites: acetoacetate, betaine, B-hydroxybutyrate (BHB), creatine, pyruvate, and valine;
b) the calculation of Logit(P₁) or Logit(P₂) logistic regressions, respectively, according to the following formulas:

−45.60+0.28E_{BHE}−4.54E_{Pyruvate}−11.23Eᵥₐₗᵢₙₑ+7.05E_{Acetoacetate}+2.85E_{creatine}−7.79E_{Betaine}; or

−42.78+0.16E_{BHB}−3.31E_{Pyruvate}−12.01Eᵥₐₗᵢₙₑ+7.0G6E_{Acetoacetate}+3.14E_{Creatine}−8.27E_{Betain}.
where E_{BHB}, E_{Pyruvate}; E_{Valine}; E_{Acetoacetate}, E_{Creatine} and E_{Betaine} correspond respectively to the measured expression levels of β-hydroxybutyrate (BHB), pyruvate, valine, acetoacetate, creatine and betaine, in the biological sample;
and
where a calculated value of Logit(P₁) or Logit(P₂) greater than 0.316 a.u. is an indication that said patient has Parkinson's disease;
where a calculated value of Logit(P₁) or Logit(P₂) lower than 0.279 a.u. is an indication that said patient does not have Parkinson's disease;
where a calculated value of Logit(P₁) less than 0.316 a.u. is an indication that said patient does not have Parkinson's disease;
where a calculated value of Logit(P₂) greater than 0.279 a.u. is an indication that said patient has Parkinson's disease.

The term «calculated value of Logit(P)» within the meaning of the present invention means a standard value obtained from the application of a logistic regression using the measurement of the levels of expression of the metabolites mentioned above. In the case of Parkinson's disease, according to the two logistic regressions above using the following 6 metabolites: acetoacetate, betaine, p-hydroxybutyrate (BHB), creatine, pyruvate, and valine, any individual obtaining a value greater than 0.316 (arbitrary unit, a.u.) will be considered to have PD and any individual scoring less than 0.279 (arbitrary unit, a.u.) will be considered not to have PD.

According to a particular mode of the method of the present invention, the biological sample of the patient comes from a patient at the prodromal or de novo stage of PD.

The term «prodromal stage of PD» means, within the meaning of the present invention, a stage where the diagnosis of PD could not be established definitively and/or with certainty, on the basis of the presented symptoms. For example, this concerns any patient presenting with non-motor symptoms frequently observed in patients who eventually develop the disease, and which could justly represent prodromal symptoms (neuropsychiatric symptoms (apathy, depression, anxiety), loss of sense of smell, constipation, sleep disorders, digestive disorders, cognitive deficits) and/or any patient who presents in his genealogical line people already suffering from the disease.

The term «de nova stage of PD» or «de novo patients» within the meaning of the present invention means newly and recently diagnosed patients who are at an early clinical stage where a neuroprotective intervention can still be envisaged to slow the progression of the disease.

According to a particular embodiment of the method of the present invention, the biological sample is for example a serum sample.

According to a particular embodiment of the present invention, the step of measuring expression levels is carried out, for example, by NMR spectroscopy measurements.

The present invention also relates to a biomarker intended for the early diagnosis of a neurodegenerative disease comprising the combination of the following at least six metabolites: acetoacetate, betaine, β-hydroxybutyrate (BHB), creatine, pyruvate, and valine.

According to a particular mode of the present invention, the biomarker further comprises the combination of at least one metabolite selected from the group consisting of alanine, lactate, DMSO2, glycine, serine, threonine, myo-inositol, and leucine.

According to a particular embodiment of the present invention, the biomarker comprises or consists of the combination of the following six metabolites: acetoacetate, betaine, β-hydroxybutyrate (BHB), creatine, pyruvate, and valine.

A subject of the present invention is also the in vitro use of a combination of metabolites according to the present invention, as a biomarker of a neurodegenerative disease, in particular of Parkinson's disease.

### BRIEF DESCRIPTION OF FIGURES

FIG. 1 represents a schematic of the experimental procedures applied to different animal models of PD. All serum samples were collected after fasting and according to the same protocol. They were analyzed by ¹H NMR at 950 MHz. Brain samples from 6-OHDA rats were used for histological analyzes and metabolomic analysis performed by HRMAS ¹H NMR at 500 MHz.

FIG. 2 represents striatal dopaminergic denervation induced by bilateral 6-OHDA substantia nigra compact (SNc) lesion resulting in apathetic-like behavior and fine motor dysfunctions. (A) PD Progression Score (PDP) used to classify 6-OHDA animals according to disease progression. The PDP score is based on the sum of the histological and behavioral components (self-administration and readjustment pitch), taking values from 0 to 4 for each. In this way, a score can be assigned to each animal from 0 (control/sham animal) to 3-7 (prodromal type animal), or 8-12 (clinical type animal). (B) Examples of coronal sections of control (sham) and 6-OHDA rat brains, stained for tyrosine hydroxylase (TH) at three representative striatal levels and localized to bregma, and corresponding patterns selected from the Paxinos atlas and Watson [78]. The areas used for quantification of dopaminergic denervation in the different analyzed sub-regions are illustrated. Scale bars represent 2 mm. (C) Quantification of the loss of TH labeling at the striatal levels indicated in (B), expressed as a percentage of the mean value obtained for the control animals (sham) (n=22). A strong decrease in TH-positive neurons in the DS and a milder decrease in the Nacc was observed in prodromal type rats (n=14) and clinical type rats (n=15). (D) 6-OHDA lesion of the SNc induced severe instrumental deficit in an operant procedure of sucrose self-administration. Mean number of sucrose doses obtained per session, (E) 6-OHDA lesion of the SNc reduced the number of readjustments in a pitch readjustment test, only in clinical type animals. Mean number of forelimb readjustments for two trials before (left bar), and after (right bar) injection of 6-OHDA (prodromal-type and clinical-type animals) or physiological saline (control/sham). Data are presented as means±SEM (standard error of the mean) and tested by one-way ANOVA or repeated measures ANOVA (RM-ANOVA), followed by Tukey's or Sidak's post-hoc test. ***: p≤0.001 compared to control (sham); ##: p≤0.01 clinical type compared to prodromal type; $$$: p≤0.0001 before surgery compared to after surgery.

FIG. 3 represents ambulatory motor activity measured in an open area. Dopaminergic lesions did not affect horizontal ambulatory activity measured over a period. Data are presented by mean value for each minute±SEM.

FIG. 4 represents the evolution of the serum metabolic profile of 6-OHDA rats modified according to the different phases of PD. (A) Example of ¹H NMR spectrum at 950 MHz (spectral areas: δ 0.5-4.7 ppm and δ 6-8.5 ppm) acquired with the CPMG (Carr-Purcell-Meiboom-Gill) pulse sequence. Assignment: 1-isoleucine; 2-leucine; 3-valine; 4-BHB; 5-lactate; 6-alanine; 7-arginine; 8-lysine; 9-acetate; 10-glutamine; 11-methionine; 12-acetone; 13-acetoacetate; 14-glutamate; 15-pyruvate; 16-citrate; 17-asparagine; 18-creatine; 19-phosphocreatine; 20-DMSO2; 21-choline; 22-phosphocholine; 23-glucose; 24-betaine; 25-myo-inositol; 26-glycine; 27-glycerol; 28-threonine; 29-glycerophosphocholine; 30-serine; 31-ascorbate; 32-glycerate: 33-proline; 34-deoxycytidine triphosphate; 35-tyrosine; 36-histidine; 37-phenylalanine; 38-formate. Macromolecules are not specified (see table 2). (B-C) OPLS (orthogonal partial least square) statistical model constructed with NMR data and animal PDP scores. R2Y=0.926; Q²=0.604; 1 predictive component and 3 orthogonal components; CV-ANOVA (analysis of variance of cross-validated residuals) p-value=3.47×10⁻⁷. (B) Graph of OPLS scores against the first predictive component and the first orthogonal component. A clear gradation of color is observed from left to right, showing that metabolic profiles change with the progression of PD. (C) Graph of OPLS weights, plotted in one dimension to mimic an NMR spectrum, with NMR variables on the abscissa. The weights are colored according to their correlation with the PDP score, from light gray (low correlation) to dark gray (high correlation). Metabolites with a correlation >0.5 are considered to be the most important in discrimination. Positive peaks indicate metabolites that are up-regulated with increasing score, while negative peaks are those that are down-regulated with changing score. (D) Relative amplitude of metabolites most involved in the metabolic gradation observed with OPLS, ie. alanine, betaine, BHB, dimethyl sulfone (DMSO2), glycine, lactate, pyruvate, serine, threonine, valine, in control animals (sham) (n=22), 6-OHDA prodromal type (n=14) and clinical type (n=15).

Mean±SEM, one-way ANOVA, followed by Tukey post-hoc test and correction for multiple comparisons. *: p≤0.05; **: p≤0.01; ***: p≤0.001.

a.u.: arbitrary units.

FIG. 5 represents the gradation of the brain metabolic profile associated with the PDP score of the animals. (A) Example of HRMAS ¹H NMR spectrum at 500 MHz. Assignment: 1-lactate allocation; 2-alanine; 3-GABA; 4-acetate; 5-glutamate; 6-glutamine; 7-N-Acetylaspartate; 8-creatine; 9-phosphocreatine (PCR); 10-phosphoethanolamine; 11-glycerophosphocholine; 12-phosphocholine (PC); 13-choline; 14-scyllo-inositol; 15-taurine; 16-myo-inositol; 17-ascorbate; 18-glycine; 19-glutathione. (B) Score plot of the OPLS model, constructed with the NMR ¹HRMAS spectra of the DS (i, top) and Nacc (ii, bottom). There is a clear gradation of colors from left to right, showing that metabolic profiles change with progression of PD, especially for DS samples (i) DS: R2Y=0.883, Q=0.703, 1+3 components, CV ANOVA p-value=0.0003; (ii) Nacc: R2Y=0.690; Q2=0.523, 1+1 components, CV ANOVA p-value=0.0005. (C) Relative amplitude of metabolites in DS (i) and Nacc (ii) in control (sham), prodromal-type and clinical-type 6-OHDA animals for the 7 key metabolites involved in the metabolic gradation observed in the OPLS, i.e. alanine, choline, glutamate, lactate, PC, PCR and taurine, Mean±SEM, one-way ANOVA, followed by Tukey post-hoc test and correction for multiple comparisons*: p≤005.

FIG. 6 represents the impact of chronic treatment with a dopaminergic (Pra) agonist on the behavioral and metabolic dysregulations of 6-OHDA rats. (A) Self-administration performance. Mean number of doses of sucrose obtained per session after the lesion or after chronic administration of a saline solution (Veh) or Pra (last 3 days). Prodromal-type animals treated with Pra show complete reversal of the effect of injury on the number of sucrose doses obtained compared to prodromal-type animals treated with Veh. Clinical-type animals treated with Pra show a moderate increase in the number of sucrose doses obtained compared to clinical-type animals treated with Veh, without achieving the performance of control animals (sham). (B) Serum metabolic deregulations. Percentage change in serum metabolites between control (sham) animals and prodromal type animals (solid bar) and between prodromal type animals treated with Veh or Pra (hatched bar). Alanine, betaine, BHB and pyruvate vary in the opposite direction to the effect of the lesion. (C) Metabolic dysregulation of the brain. Percentage change in brain tissue metabolites normalized to Veh-treated control (sham) animals in DS and Nacc for prodromal type (i) and clinical type (ii) animals. Animals treated with pramipexole (hatched bar) showed an inverse variation in dysregulation of metabolite levels compared to animals treated with Veh (solid bar). Mean±SEM, one-way ANOVA, followed by Tukey post-hoc test, **: p≤0.01; ***p≤0.001; #: p≤0.05.

FIG. 7 represents the absence of metabolic dysregulation at the serum level in the animals having received an infusion of comparison group virus GFP (Green fluorescent protein). Histogram representing the levels of the metabolites corresponding to the different samples taken from GFP animals for acetoacetate, betaine, BHB, creatine, glycine, myo-inositol, pyruvate, and serine which represent the main metabolites involved in the discrimination of the 3 groups in OPLS-DA (discriminant analysis) of α-synuclein animals. White bars correspond to samples at week 0, light gray bars at 3 weeks after virus infusion and dark gray bars at 10 weeks after. Data are presented as mean value±SEM and are tested by one-way ANOVA, followed by a Tukey post-hoc test.

FIG. 8 represents the metabolomic study by NMR on serum samples of the other models of PD in different species (A) α-synuclein rat animal model. OPLS-DA scores constructed from ¹H NMR spectra of serum, compared to the first 2 orthogonal predictive components. The 3 groups, mimicking the 3 different phases of PD, are clearly discriminated. R2Y=0.929; Q2=0.518; 1 predictive component and 3 orthogonal components; CV ANOVA p-value=0.008. (B) Relative amplitude of the metabolites most involved in the discrimination between α-synuclein animals of prodromal type (n=11), clinical type (n=11) and control animals (sham) (n=12) i.e. acetoacetate, betaine, BHB, creatine, glycine, myo-inositol (myo), pyruvate, serine. (C) MPTP non-human primate model. OPLS-DA scores constructed from ¹H NMR spectra of serum, relative to the first 2 predictive and orthogonal components. Control animals (sham) and clinical type animals are clearly differentiated. R2Y=0.998; Q²=1 predictive component and 1 orthogonal component; p-value of ANOVA CV=(D) Histogram showing relative quantitative changes in metabolites between MPTP (n=8) and control (sham) animals (n=6) for acetoacetate, alanine, betaine, BHB, creatine, lactate, pyruvate and valine, representing the main metabolites involved in the discrimination of the 2 groups in OPLS-DA. Mean±SEM, mixed model followed by Tukey post-hoc test or Mann-Whitney t-test *: p≤0.05; **: p≤0.01; ***: p≤0.001.

FIG. 9 represents the effect of pramipexole on BHB and betaine levels in PD patients in the US cohort. Histogram showing the relative quantitative values of metabolites in the control group (comparison group) (white bar), patients with PD (dark bar) and patients with PD and treated with Pra (hatched bar). Data are presented as mean value±SEM.

FIG. 10 represents the logistic regression having made it possible to construct a composite biomarker from 6 serum metabolites: BHB, acetoacetate, valine, creatine, betaine and pyruvate. (A) ROC (Receiver operating curve) curve with serum sample data from control (sham) and prodromal-type (6-OHDA and α-synuclein rats) animal models (AUC (area under the curve)=0.936, sensitivity: 0.853, specificity: 0.88). (B) ROC curve with data from PD patients and corresponding comparison groups from the US cohort (AUC=0.88, sensitivity: 0.957, specificity: 0.714).

FIG. 11 represents the logistic regression which made it possible to construct a composite biomarker from 6 serum metabolites: BHB, acetoacetate, valine, creatine, betaine and pyruvate. ROC Curve with Data from PD Patients and corresponding comparison groups from US cohort (minus 2 patients not selected) (AUC=0.88, sensitivity: 0.957, specificity: 0.714) and from the Italian cohort (AUC=sensitivity: 0.65, specificity: 0.90).

FIG. 12 represents the logistic regression curve for the panel of serum metabolites including: BHB, acetoacetate, valine, creatine, betaine and pyruvate. (A) ROC curve from serum samples of all MP type animal models. AUC=0.954, sensitivity: 0.85, specificity: 0.915. (B) Logistic regression algorithm for all PD type animals compared to the control group (sham).

### EXAMPLES

### Example 1: Materials and Methods

Scheme

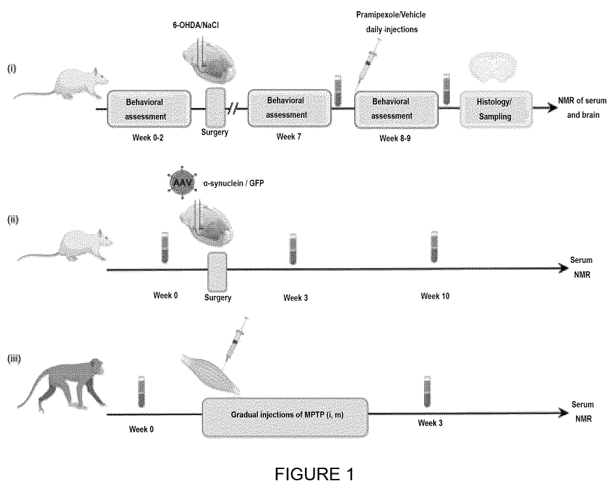

FIG. 1 illustrates a schematic of the entire protocol applied for animal models. Rats from the first cohort (FIG. 1(i)) were trained for two weeks on a sucrose self-administered task until they achieved stable performance and were subjected to a pitch readjustment test before receiving a bilateral intracerebral injection of 6-OHDA (n=29) or physiological saline (NaCl, n=22) into the SNc. After lesion stabilization (approximately 3 weeks), self-administration was resumed for one week, and the pitch readjustment test was repeated to monitor changes in animal performance, before each group was subjected (6-OHDA and NaCl) to 2 weeks of daily injections of Pra 0.2 mg/kg (6-OHDA Pra n=15; NaCl Pra n=11) or physiological serum (NaCl 0.09%; 6-OHDA NaCl n=14; NaCl NaCl n=11). Self-administration was continued throughout Pra treatment and a final pitch readjustment test was performed at the end of treatment. Serum samples were collected after intracerebral injection of 6-OHDA or NaCl, following stabilization of self-administration performance and at the end of Pra treatment. The brains were collected at the end of the behavioral procedure, frozen in liquid nitrogen and kept at −80° C. before being processed for histology and HRMAS ¹H NMR experiments.

The second cohort of rats (FIG. 1(ii)) received an intracerebral injection of AAV-hA53Tα-syn (n=12) or AAV-GFP (n=12) into the SNc. Serum samples were collected longitudinally during the study, before, 3 and 10 weeks after AAV injection.

Monkeys (n=8) received intramuscular injections of MPTP (0.3 to 0.5 mg/kg) every 4-5 days for 3 weeks [77]. The repeated administration of low doses of MPTP has been used to mimic a moderate stage, with slow disease development, and the onset of moderate dopaminergic injury. Serum samples were collected at the start of the experiment and after the monkeys reached a stable parkinsonian state (FIG. 1(iii)).

All serum samples were analyzed by ¹H NMR at 950 MHz and brain samples were subjected to HRMAS ¹H NMR at 500 MHz.

Animals

Rats: the experiments were carried out on adult male Sprague-Dawley rats (Janvier, Le Genest-Saint-Isle, France), weighing approximately 300 g (7 weeks) at the start of the experiment, They were housed under standard laboratory and ethical conditions with an inverted light-dark cycle (12 p.m./light-dark cycle, with lights on at 7 p.m.) and with food and water available ad libitum.

Monkeys: the experiments were carried out on adult male Macaca fascicularis, weighing between 5 and 8 kg, aged 3 to 5 years and housed under standard conditions (light-dark cycle of 12:12 hours; 23° C.; 50% humidity).

The used protocols complied with the European Union Animal Protection Act 2010 and French Directive 2010/63, and were approved by the French National Ethics Committee (2013/113) No. 004 and by the local ethics committee C2EA84 and CELYNE C2EA.

Surgery

Bilateral injection of 6-OHDA: As described previously [18, 23], the rats received a subcutaneous injection of desipramine (15 mg/kg) 30 minutes before the injection of 6-OHDA in order to protect the noradrenergic neurons. All animals were then anesthetized by intraperitoneal injection of ketamine-xylazine (100-7 mg/kg), and placed in a stereotaxic frame (Kopf Instruments). The stereotaxic coordinates of the injection sites were as follows, according to the stereotaxic atlas of Paxinos and Watson [78] and in relation to the bregma: incisor bar placed at −3.2 mm: anteroposterior (AP)=−5.4 mm/lateral (L)=+/−1.8 mm/dorsoventral (V)=−8.1 mm. The animals received a bilateral injection of 2.3 μl of 6-OHDA (3 mg/ml) or physiological saline (NaCl 0.9%; control/sham), at a flow rate of 0.5 μl/min. For animals injected with 6-OHDA and presenting with transient starvation 2-3 days after the operation, supplementation with a high-calorie liquid diet and appetizing foods was started for 1-2 weeks, and stopped 10 days before serum collection. Animals that did not recover were excluded.

Bilateral injection of α-synuclein: As previously described [19], rats were anesthetized with 2.5% isoflurane and placed in a stereotactic frame (Kopf Instruments) in order to receive two bilateral injections of AAV-hA53Tα-syn (1 μl-7.0×10¹² vg/ml) or AAV-GFP (7.0×10¹² vg/ml, control/sham animals) in the SNc (AP=−5.1 and −5.6 mm/L=+1-2.2 mm/V=8 mm from the bregma). After being awakened, the animals were returned to the animal facility.

Behavioral Assessment

6-OHDA rats: The rats were subjected to 2 behavioral tests, 3 weeks after the operation, while the 6-OHDA lesion was stabilized [79].

Operant self-administration of sucrose (motivational component): Rats were trained to self-administer a 2.5% sucrose solution in operant chambers (Med Associates, St Albans, VT, USA) containing an active lever, reinforced, for which pressing causes the delivery of 0.2 ml of a sucrose solution associated with a low-intensity light stimulus, and an inactive, unreinforced lever, for which pressing causes neither delivery of sucrose nor light stimulus. This task was performed with a fixed ratio of 1, with a single press of the reinforced lever resulting in the delivery of a reward. Each session ended when 100 rewards were obtained, or at the end of the allotted time (1 hour). The number of obtained rewards was counted for each session by the MED-PC IV software.

Pitch readjustment test (motor component): The animals, held by the posterior third of their body, were moved over a length of 90 cm by a straight and regular movement from left to right, and vice versa, along a smooth surface table. The number of forelimb adjustments made during movement was counted [80]. The test was carried out three times, by two different experimenters, blind (ignorance of the nature of the experimental groups).

MPTP monkeys: The severity of the parkinsonian state was assessed using a rating scale taking into account classic motor symptoms (bradykinesia, rigidity, tremors, freezing, posture and position of the arms), spontaneous activities (arm movements, spontaneous eye movements and activity in the housing cage) and other activities (vocalization, triggered eye movements and feeding) [81].

Histology: Immunolabeling of TH and Quantification of Denervation in 6-OHDA Rats.

Brain harvest: The rats were sacrificed by decapitation 1 day after the last session of sucrose self-administration and 1 hour after the last injection of Pra or Veh (that is to say 6 weeks after the injection of 6-OHDA or saline solution). The brains were immediately frozen in liquid nitrogen and stored at −80° C. They were then sliced using a cryostat thermostated at −20° C. Sections of the regions of interest were taken to carry out both the immunostaining of TH and to carry out the metabolomic study. For ¹HHRMAS NMR, thick sections of DS and Nacc tissue were pooled in inserts, and kept at −80° C. until NMR analysis.

For TH immunostaining, 14 μm coronal sections of the striatal levels of interest were taken and stored at −20° C. Immunostaining was then performed as previously described [18,23]. Briefly, after fixation with 4% paraformaldehyde, striatal sections were incubated with an anti-TH antibody (mouse monoclonal MAB5280, Millipore, France, 1:2500) overnight at 4° C. Then, the sections were incubated with a biotinylated goat anti-mouse IgG antibody (BA-9200, Vector Laboratories, Burlingame, CA, USA; 1:500) and the immunoreactivity revealed by the avidin-peroxidase conjugate (Vectastain ABC Elite, Vector Laboratories Burlingame, CA, USA).

Quantification of the extent of striatal dopaminergic denervation was determined with the computerized image analysis system ICS FrameWork (Calopix, version 2.9.2, TRIBVN, Châtillon, France) coupled to an optical microscope (Nikon, Eclipse 80i). After drawing the masks of the three striatal levels of interest, the optical densities (OD) were measured for each striatal subregion (DS and Nacc). The ODs were expressed as percentages relative to the mean optical density obtained from the homologous regions of the operated control animals (intracerebral injection of physiological saline).

Progression of Parkinson's Disease (PD) Score in the 6-OHDA Model

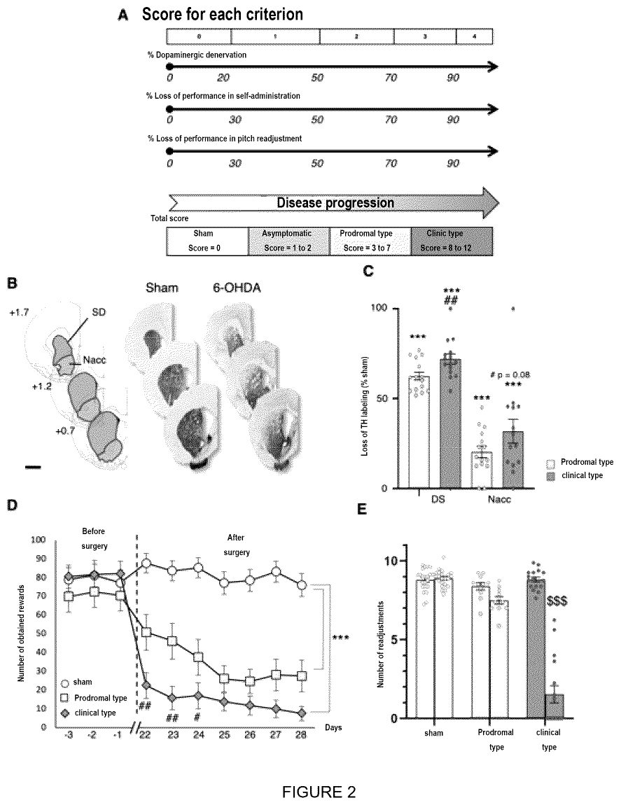

In order to assess the progression of PD (PDP) in 6-OHDA rats, a scale called «PDP score», and combining 3 criteria, was developed: 1) criterion 1: assessment of neuropsychiatric symptoms (ie. behavior apathetic type), 2) criterion 2: assessment of motor symptoms (i.e. fine motor deficit), these two criteria also being used clinically in the scales allowing the diagnosis of PD, and 3) criterion 3: the level of dopaminergic denervation in the DS, available only after postmortem histological analysis in the patient. Each criterion was quantified for each animal, with values ranging from 0 to 4, as shown in FIG. 2A. The values assigned to each criterion were added together to produce the PDP score, ranging from 0 to 12.

For the assessment of behavioral deficits in the self-administration and pitch readjustment tests (criteria 1 and 2, respectively), the performance of each animal was compared before and after the injection of 6-OHDA (or physiological saline (sham). A value of 0 for each of these two criteria corresponded to an absence of deficit, or to a deficit of less than 30%, i.e. the normal daily variability of the used tests. The following subcategories have been set as follows: 1=low deficit (30%-50%); 2=mean symptoms (50%-70%); 3=strong symptoms (70%-90%); 4=significant or total deficit (>90%).

Considering that motor symptoms appear after 70% loss of striatal dopaminergic neurons in patients with PD, the value of dopaminergic denervation in DS (criterion 3) was used to define the boundary between the prodromal phase and the clinical phase.

With regard to the PDP score obtained by adding the values of the 3 criteria, the animal mimicking PD was classified as: 1) asymptomatic rat with weak lesions but no behavioral symptoms (PDP score=1 to 2), 2) prodromal type animal, i.e. presenting only neuropsychiatric disorders and a small DS lesion (PDP score=3 to 7) and 3) clinical type animal, i.e. presenting neuropsychiatric disorders; motor symptoms and an extensive DS lesion (PDP score=8 at 12). Control (sham) animals were assigned a score of 0.

Given the low number of asymptomatic animals and the fact that they are of no clinical relevance, they were excluded from the study.

Human Cohorts

Blood samples from PD patients and corresponding comparison groups subjects were obtained through 1) the Parkinson's Disease Biomarker Program (PDBP) Consortium, supported by the National Institute of Neurological Disorders and Vascular Accidents National Institutes of Health (NIH-USA), and 2) Santa Lucia Foundation (Rome, Italy). All subjects (patients and comparison groups) gave written informed consent. Inclusion criteria for patients from the United States included recent diagnosis (<1 year) of PD according to the new Movement Disorders Society (MDS) criteria. For the Italian cohort, the diagnostic criteria were less restrictive and patients diagnosed for longer than those of the US cohort were included (0 to 3 years after diagnosis), In both cohorts, the patients had not received any antiparkinsonian treatment at the time of inclusion, did not have dementia or psychiatric disorders, or any other medical condition that could compromise the study. Inclusion criteria for comparison groups were: absence of neurological disease, absence of family history of movement disorders, and absence of specific medical conditions. The US cohort included 21 PD patients without antiparkinsonian therapy, 9 PD patients who received Pra treatment, and 30 age- and gender-matched comparison groups. The Italian cohort included 21 patients with PD and 23 age- and gender-matched comparison groups. All clinical and demographic information collected from the two human cohorts has been summarized in Table 1 below (data are represented by mean value±SEM, M: Male; F: Female; *2 patients of unknown sex),

TABLE 1

| | NIH | | Italy | |
|---|---|---|---|---|
| | Control | PD | Control | PD |
| Age | 61.4 ± 1.8 | 62.0 ± 2.1 | 57.26 ± 1.89 | 60.38 ± 2.00 |
| Gender | 16 M/14 F | 9 M/12 F | 9 M/14 F | 14 M/7F* |

NMR Experiments

HRMAS ¹H NMR of 6-OHDA rat brain samples: Just before HRMAS analysis, 10 μl of D₂O was added to the inserts containing the brain biopsies, which were then sealed and placed in a 4 mm zirconium rotor.

Data acquisition: All HRMAS NMR spectra were acquired, as previously described [82], on a Bruker Advance Ill spectrometer (Billerica, USA) (IRMaGe, CEA Grenoble, France) at 500 MHz. The samples were rotated at 4000 Hz at the magic angle (54.7°) and the temperature was maintained at 4° C. for all experiments. One-dimensional spectra were acquired using a CPMG pulse sequence (TE=30 ms, 256 repetitions, 17 min). The residual water signal was pre-saturated for 1.7 s of relaxation.

Data processing: Quantification was performed with JMRUI software using quantification based on a quantum estimation procedure (QUEST) [83]. This procedure requires the use of a database of metabolites and a complete assignment of spectra. Nineteen metabolites were attributed and quantified: acetate, alanine, ascorbate, choline, creatine, gamma-aminobutyrate (GABA), glutamate, glutamine, glycine, glycerophosphocholine, glutathione, lactate, myo-inositol, N-acetylaspartate, phosphocholine, phosphocreatine, phosphoethanolamine, scyllo-inositol and taurine. The amplitude of the metabolite calculated by QUEST was normalized with respect to the total signal of the spectrum. CRLBs were calculated for each metabolite as an 2.5 estimate of the standard deviation of the fit.

¹H NMR of Serum

Serum collection: For the rats, the blood was collected from the tail vein after 2 hours of fasting and under gaseous anesthesia with isoflurane (2%) and was stored in ice before being quickly centrifuged at 1600 g for 15 min at 4° C. The supernatant serum was collected and stored at −80° C. until the day of the NMR. The time before freezing never exceeded 30 min [84].

For monkeys, blood was collected from the saphenous vein after fasting for 12 hours, under anesthesia (atropine 0.05 mg/kg i.m. followed by Zoletil 15 mg/kg i.m.) and subjected to the same protocol as rat blood.

For humans, blood was collected after overnight fasting, stored at room temperature for 15-60 min before being centrifuged at 1200-1500 g at 4° C. for 10-15 min. The supernatant serum was then collected and stored at −80° C.

On the day of the NMR experiments, the samples were slowly thawed on ice and then quickly centrifuged to remove any cryo-precipitates. NMR tubes were filled with 60 μl of serum sample and 120 μl of 0.1 M phosphate buffered saline (PBS) in D₂O (50% D₂O, pH=7.4), and stored at 4° C. until acquisition by NMR.

Data acquisition: All animal and human serum samples were subjected to the same NMR protocol. ¹H NMR experiments were performed on a Bruker Advance III NMR spectrometer at 950 MHz (IBS, Grenoble, France) by using a 3 mm cryoprobe. Spectra were recorded using a CPMG pulse sequence. The residual water signal was pre-saturated for 2 seconds of relaxation.

Peak assignment was performed using the 2-dimensional homonuclear ¹H-¹H (TOCSY) and heteronuclear ¹H-¹³C (HSQC) experiments, and databases. If necessary, addition of selected metabolites was used to clarify ambiguous identifications.

Data processing: The time signals were transformed by the Fourier method and phased manually with Topspin software from Bruker, version 3.6.2. Then, other pre-processing steps (baseline correction, alignment, segmentation) were performed using the online NMRProcFlow v1.2 software (http://nmrprocflow.org). Segments of ppm were made over the 0-10 ppm spectral range, excluding the residual water peak, macromolecule/lipid signals and other regions containing pollutions (at δ 5.0-4.7; δ 5.25-5.38; δ 2.2-2.0; δ 1.4-1.25; δ 1.15-1; δ 0.9-0.5). Each segment was normalized to the total sum of the segments.

Statistical Analysis

Multivariate analysis: Data from liquid NMR or HRMAS were imported into SIMCA v.14 (Malmo, Sweden) for multivariate statistics. Unsupervised principal component analysis (PCA) was first used for the overall visualization of the distribution of all samples, followed by OPLS to find the discriminating metabolites associated with a specific disease stage. For the latter, either a continuous variable (that is to say the PDP score), either a discrete variable (that is to say group membership) was used to label each sample, leading to OPLS-DA in the latter case. The total number of components was determined using the cross-validation procedure, which produces the R2Y and Q2 factors that indicate the goodness of fit and the predictability of the model, respectively. A model is considered robust and predictive when both factors are ≥0.5. The scores were plotted in 2D against the first two components of the OPLS models, while the weights were plotted in 1D to mimic an NMR spectrum but with positive/negative peaks indicating up-regulated/down-regulated metabolites, respectively. Indeed, in this «statistical spectrum», the correlation of each NMR variable with the continuous ranking variable (score), ranging from 0 to 1, was color coded, ranging from dark gray (high correlation) to light gray (low correlation). Metabolites with a correlation ≥0.5, considered the most discriminating, were subjected to univariate analysis. Analysis of variance of cross-validated predictive residuals (CV-ANOVA) was used to assess the significance of the model.

Metabolites significantly varying in at least three of the four studied models (6-OHDA, α-synuclein, MPTP and human) were subjected to multiple logistic regression to assess their relevance as a potential predictive biomarker of PD at using the R software (version 3.6.1—R core team). The regressions, including cross-validation, were performed for prodromal type animals (6-OHDA and α-synuclein prodromal type rats). Clinical-type animals were used for external validation (clinical-type 6-OHDA and α-synuclein rats, and all primates). The same analyzes were performed for the US patient cohort. The Italian patient cohort was used for external validation. All combinations of retained metabolites were tested, and ROC curves were then generated for each of them to assess the goodness of fit using the area under the curve (AUC) and the optimal threshold that maximizes sensitivity and specificity.

Univariate Analysis:

All univariate analyzes were performed using Graphpad Prism 8 software (San Diego—USA). All results were expressed as mean values±SEM with a significance level set at 0.05.

6-OHDA models: For the self-administration tests and the step readjustment tests, a univariate analysis of the effect of Pra was performed by applying a one-way repeated measures (MR) ANOVA followed by a Sidak post-hoc test.

For histological and metabolomic data, a one-way ANOVA followed by Tukey post hoc test with correction for multiple comparisons was performed.

α-synuclein models: For the longitudinal study performed in α-synuclein animals, some values were missing, due to artifacts during NMR measurement, Data were therefore analyzed by fitting a proposed mixed model using a compound symmetry covariance matrix, and were fitted using the restricted maximum likelihood (REML) method. This model was followed by a Tukey post hoc test.

MPTP monkeys: Given the small number of animals, the non-parametric Mann-Whitney test was used.

Humans; Data were log-transformed given the non-Gaussian distribution of metabolite levels. First, t-tests were performed individually in the two different cohorts. Then a two-way ANOVA was applied with i) origin of cohort and ii) group (comparison group or PD) as factors, followed by Sidak post hoc test.

### Example 2: Results

The 6-OHDA Rat Model Makes it Possible to Study the Different Stages of Parkinson's Disease

Although, in humans, the diagnosis of PD relies on a scale that incorporates assessments of neuropsychiatric and motor symptoms [22], its definitive confirmation is based on postmortem histological assessments, primarily of neuronal loss in the nigrostriatal pathway leading to DS denervation. To characterize 6-OHDA rats for clinical disease progression, a score, called the Parkinson's disease progression score (PDP), based on the same type of criteria as those used in humans, ie. the neuropsychiatric component—assessed by self-administration performance (motivation)—, fine motor skills—assessed by the performance of the step readjustment test—, and the extent of the DS lesion—assessed by the post-mortem histological analysis was assigned to them. Based on this score, animals with PD were divided into two categories, namely prodromal type animals and clinical type animals.

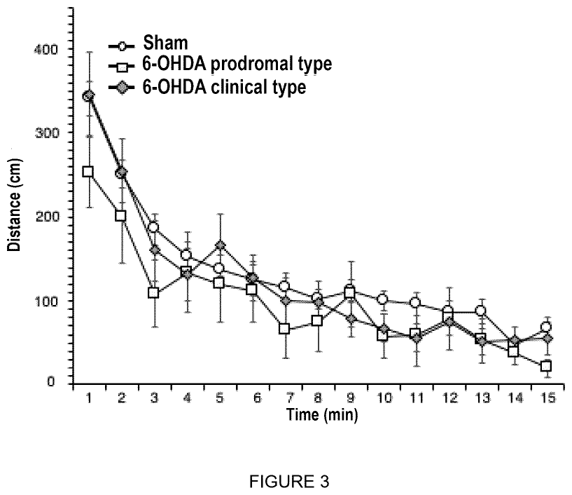

Tyrosine hydroxylase immunoreactivity (TH-IR) revealed that bilateral injection of 6-OHDA into the SNc led to partial nigrostriatal dopaminergic injury resulting in dopaminergic denervation in the DS and to a lesser extent in the Nacc (FIGS. 2B and 2C). Indeed, TH-IR quantification showed a strong loss (62.2%) of dopaminergic projections in the DS of prodromal-type animals compared to controls (sham), an even greater loss in clinical-type animals (71.96%). A slight loss of TH-IR was also observed in the Nacc of 6-OHDA animals (FIG. 2C). This pattern of denervation preserves learning abilities and overall ambulatory activity of animals [18], allowing the study of motivational processes, without the potential bias related to locomotor alterations often present in animal models (FIG. 3) [18, 23]. The motivation was measured by the sucrose self-administration procedure. Prior to the injection of 6-OHDA (saline for shams), the rats were trained on the self-administration task, which allowed them to reach their maximum level of performance (approximately 80 rewards per one hour training session). During the post-injury phase (i.e. after 6-OHDA/physiological saline injection), the performance of control rats (sham, saline injection) remained stable, while that of 6-OHDA animals decreased significantly [30-66%] (FIG. 2D).

With respect to fine motor skills, all animals showed a similar number of paw readjustments before 6-OHDA/physiological saline injection, which was not changed after 6-OHDA injection, except in the clinical type group where it was significantly reduced (FIG. 2E).

Co-Evolve Serum Metabolic Signatures with PD Progression in 6-OHDA Rats

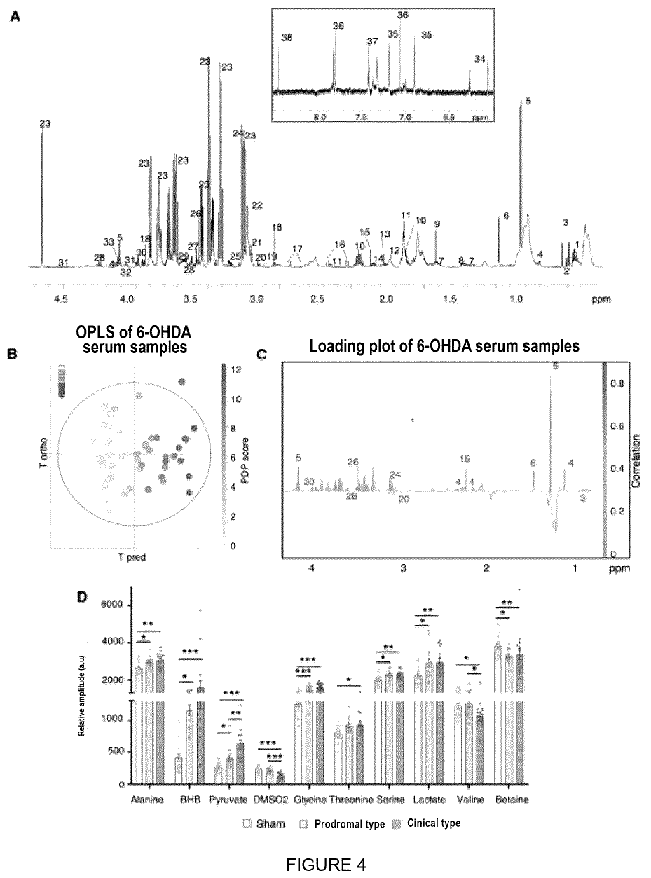

FIG. 4A illustrates a typical ¹H NMR spectrum of 6-OHDA rat serum, acquired at a very high magnetic field (23 T). This allowed the identification of approximately 50 metabolites in the serum samples. For each compound, the assignment ¹H and ¹³C of their chemical groups, as well as the multiplicity of the peaks, are presented as follows: singlet (s), doublet (d), doublet doublet (dd), multiplet (m).

TABLE 2

| | | ¹H | ¹³C | |
|---|---|---|---|---|
| Metabolite | Groupe | ppm | ppm | Multiplicity |
| 3- | γ-CH₃ | 1.19 | 24.4 | d |
| hydroxybutyrate | half α-CH₂ | 2.30 | 49.2 | dd |
| | half α-CH₂ | 2.39 | 49.2 | dd |
| | β-CH | 4.15 | | m |
| Acetoacetate | | 3.44 | | s |
| | | 2.27 | | s |
| Acetate | CH₃ | 1.91 | 25.9 | s |
| Acetone | CH₂CO | 2.22 | 32.9 | s |
| Alanine | CH₃ | 1.47 | 18.8 | d |
| | α-CH | 3.77 | 53.5 | q |
| Albumin lysyl | ε-CH₂ | 2.88 | 42.0 | t |
| | ε-CH₂ | 2.95 | 41.9 | t |
| | ε-CH₂ | 3.01 | 42.0 | t |
| Arginine | γ-CH₂ | 1.64 | | |
| | γ-CH₂ | 1.69 | | |
| | β-CH₂ | 1.89 | | |
| | δ-CH₂ | 3.23 | 43.2 | t |
| Aspartate | half β-CH₂ | 2.66 | | q |
| | half β-CH₂ | 2.80 | | dd |
| Betaine | CH₂ | 3.9 | | s |
| | CH₃ | 3.26 | | s |
| Cholesterol | C18 (in HDL) | 0.66 | | m |
| | C18 (in VLDL) | 0.68 | | |
| | C2 [symbol] and C27 | 0.83 | 25.2 | m |
| Choline | N (CH₃)₃ | 3.21 | 56.7 | s |
| | | 3.51 | | |
| | | 4.06 | | |
| Citrate | half CH₂ | 2.53 | | d |
| | half CH₂ | 2.66 | | d |
| Creatine | CH₃ | 3.03 | | s |
| | CH₂ | 3.92 | | s |
| Creatinine | CH₃ | 3.04 | | s |
| | CH₂ | 4.05 | | s |
| Dimethylamine | CH₃ | 2.71 | | s |
| Ethanol | CH₃ | 1.17 | 21. [symbol] | t |
| | CH₃COH | 3.65 | | q |
| Fatty acids | CH₃(CH₂)n | 0.84 | 16.5 | m |
| (mainly LDL) | (CH₂)n | 1.27 | 32.2 | m |
| Fatty acids | CH₃CH₂CH₂C═ | 0.86 | | m |
| (mainly VLDL) | CH₂CH₂CO | 1.57 | 27.4 | m |
| | CH₂CH₂CH₂CO | 1.29 | | m |
| Fatty acids | Ch₃CH₂ | 0.93 | 21.05 | m |
| | CH₃CH₂(CH₂)n | 1.24 | 34.4 | m |
| | CH₃CH₂(CH₂)n | 1.26 | 25.2 | m |
| | CH₂ | 1.26 | 19.2 | m |
| | CH₂ | 1.30 | | m |
| | CH₂CH₂C═C | 1.68 | 29.2 | |
| Fatty acid | CH₂C═C | 2.00 | 29.7 | m |
| | CH₂CO | 2.22 | 3 [symbol] .3 | m |
| | C═CCH₂C═C | 2.72 | 28.1 | m |
| | CH═CHCH2CH═CH | 5.26 | 10. [symbol] | m |
| | CH═CHCH2CH═CH | 5.29 | 132.2 | m |
| Formate | CH | 8.45 | | s |
| Fructose | | 3.99 | | m |
| | | 4.01 | | dd |
| Fucose/β- | | 4.54 | | d |
| Galactose | | | | |
| Glucose | H2 | 3.24 | 7 [symbol] .9 | t |
| | H4 | 3.40 | 72.4 | t |
| | H4 | 3.41 | 72.4 | t |
| | H5 | 3.4 [symbol] | 78. [symbol] | m |
| | H3 | 3.48 | 78.5 | t |
| | H2 | 3.53 | 74.3 | q |
| | H3 | 3.71 | 75.6 | t |
| | half CH₂—C6 | 3.72 | 63.5 | q |
| | half CH₂—C6 | 3.76 | 63.4 | m |
| | H5 | 3.82 | 74.2 | ddd |
| | half CH₂—C6 | 3.84 | 63.4 | m |
| | half CH₂—C6 | 3.89 | 63.5 | dd |
| | H1 | 4.64 | 98.7 | d |
| | H1 | 5.23 | 94.9 | d |
| Glutamate | half β-CH₂ | 2.04 | | m |
| | half β-CH₂ | 2.12 | | m |
| | half γ-CH₂ | 2.34 | 33.9 | m |
| | half γ-CH₂ | 2.3 [symbol] | | m |
| | | 3.74 | | m |
| Glutamine | | 2.08 | | |
| | | 2.09 | | |
| | half β-CH₂ | 2.11 | 29.7 | m |
| | half γ-CH₂ | 2.44 | 33.9 | m |
| | | 2.4 [symbol] | 57.4 | m |
| | | 3.74 | | |
| Glycerol | half CH₂ | 3.5 [symbol] | 65.8 | q |
| | half CH₂ | 3.65 | 65.6 | q |
| | C₂—H | 3.87 | 74.6 | m |
| Glyceropho- | | 3.22 | | s |
| sphocholine | NCH₂ | 3.66 | 68.7 | m |
| | OCH₂ | 4.29 | 62.2 | m |
| Glycerol | | 4.06 | | |
| backbone | | | | |
| PGLYS and | CHOCOR | 4.22 | | |
| TAGS | | 5.20 | | |
| Glycine | CH₂ | 3.55 | 44.3 | s |
| Histidine | | 3.09 | | dd |
| | | 3.98 | | dd |
| | H4 | 7.04 | | s |
| | H2 | 7.75 | | s |
| Isoleucine | δ-CH₃ | 0.93 | 13.9 | t |
| | β-CH₃ | 1.00 | 17.15 | d |
| | half γ-CH₂ | 1.24 | | |
| | half γ-CH₂ | 1.46 | | |
| | | 1.96 | | |
| | | 3.65 | | |
| Lactate | CH₃ | 1.32 | 22.7 | d |
| | CH | 4.11 | 71.2 | q |
| Lactose | | 3.55 | | |
| | | 3. [symbol] 6 | | |
| | | 3.97 | | |
| | | 4.45 | | |
| Leucine | δ-CH₃ | 0.95 | | d |
| | δ-CH₃ | 0.96 | | d |
| | | 1.66 | | m |
| | | 1.70 | 42.7 | m |
| | | 1.73 | | m |
| | α-CH | 3.71 | | |
| Lysine | γ-CH₂ | 1.43 | | m |
| | γ-CH₂ | 1.49 | | m |
| | δ-CH₂ | 1.72 | | m |
| | β-CH₂ | 1.88 | | m |
| | β-CH₂ | 1.91 | | m |
| | | 3.02 | | t |
| | | 3.74 | | t |
| Mannose | | 4.89 | | d |
| | | 5.18 | | d |
| Methanol | CH₃OH | 3.35 | | s |
| Methionine | | 2. [symbol] 4 | 31.4 | t |
| Methionine | | 2.15 | | s |
| Myo-inositol | | 4.05 | | |
| | | 3.27 | | t |
| N-acetyl- | NHCOCH₃ | 2.04 | 24.7 | s |
| glycoprotein 1 | | | | |
| N-acetyl- | NHCOCH₃ | 2.07 | 25 | s |
| glycoprotein 2 | | | | |
| Phenylalanine | half β-CH2 | 3.26 | | |
| | α-CH | 3.97 | | |
| | H2, H6 | 7.31 | | d |
| | H4 | 7.35 | | m |
| | H3, H5 | 7.40 | | m |
| Proline | γ-CH₂ | 1.98 | | m |
| | γ-CH₂ | 2.01 | | m |
| | half β-CH2 | 2.05 | | m |
| | half β-CH2 | 2.34 | | m |
| | half δ-CH2 | 3.33 | | m |
| | α-CH | 4.12 | | m |
| Succinate | | 2.39 | | s |
| Threonine | γ-CH₃ | 1.31 | | d |
| | α-CH | 3.55 | | d |
| | β-CH | 4.23 | | m |
| Trehalose | | 3.40 | 74.3 | |
| Tyrosine | | 6.88 | | d |
| | H2, H6 | 7.18 | | d |
| Valine | CH3 | 0.98 | 19.4 | d |
| | CH3 | 1.03 | 20.6 | d |
| | α-CH | 2.26 | | m |
| | β-CH | 3.60 | 63.4 | d |
| Urea | NH₂C═ONH₂ | 5.77 | | |
| Xylose | | 3.41 | 78.6 | |

[symbol]

indicates data missing or illegible when filed

The graph of the OPLS scores, made with the NMR data and the PDP scores for each animal (i.e. the «6-OHDA serum OPLS model») shows a clear gradation of the dot colors, from «cold» (white) on the left to «warm» (dark gray) on the right. This indicates that the metabolic profile is changing along with disease progression (FIG. 4B). Among the 14 most relevant metabolites (i.e. correlation with the PDP score ≥0.5), ten of them were significantly modified in at least one PD type group compared to control animals (sham) (FIG. 4C): alanine, betaine, B-hydroxybutyrate (BHB), dimethyl sulfone (DMSO2), glycine, lactate, pyruvate, threonine, serine and valine. These metabolites did not evolve identically with the progression of the disease. First of all, 6 metabolites were significantly modified from the «prodromal» phase: alanine, BHB, glycine, lactate and serine increased, while betaine decreased. Their levels then remained stable in the «clinical» phase. Then, a metabolite, pyruvate, gradually increased according to the different phases of PD. Finally, three metabolites were only changed at the clinical stage: DMSO2 and valine decreased, while threonine increased (FIG. 4D).

Metabolic Profiles of Brain Tissue in the 6-OHDA Rat Model Strengthen Blood Findings

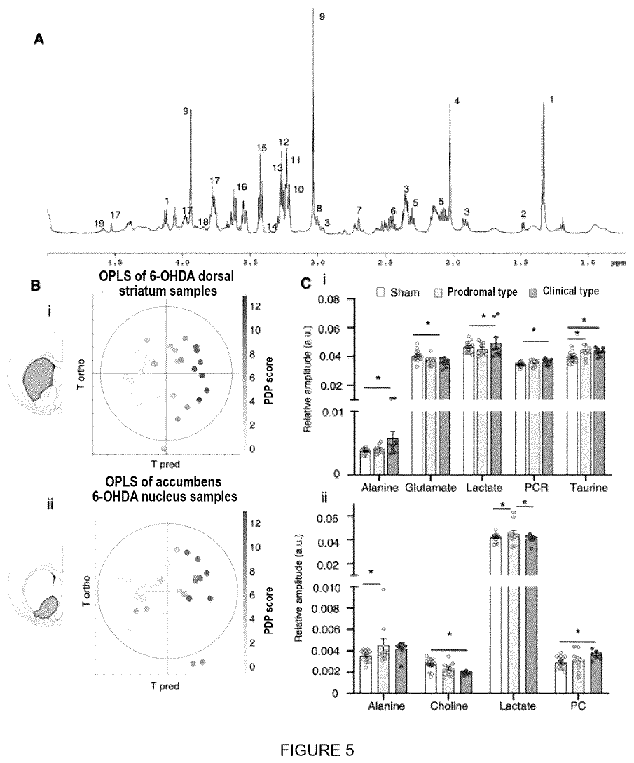

The «High Resolution Magic Angle Spining» (HRMAS) ¹H NMR” spectroscopy was used to study the metabolic profiles of intact brain biopsies (DS and Nacc) of 6-OHDA animals, sampled at the same level as sections used for histology to quantify dopaminergic denervation. A representative spectrum of a DS biopsy from a 6-OHDA rat is shown in FIG. 5A, with the labeling of assigned and quantified metabolites. With respect to DS samples, the score plot of the OPLS analysis (i.e. the «OPLS 6-OHDA DS model») revealed a co-evolution of brain metabolic profiles with the animals PDP score, as in serum (FIG. 5B(i)). Significant dysregulations of major metabolites are illustrated in FIG. 5C(i). Compared to control (sham) animals, a metabolite, taurine, was significantly altered in the prodromal type group (increase), while four metabolites were significantly altered in the clinical type group: alanine, lactate and phosphocreatine increased and glutamate decreased.

The OPLS analysis constructed with the Nacc data (i.e. the «OPLS 6-OHDA Nacc model») showed the same trend, although less clear (FIG. 5B(ii)), Univariate statistics revealed a significant increase in alanine and lactate in the prodromal-type group, whereas the clinical-type group was characterized by a significant increase in phosphocholine (PC) and a decrease in choline compared to control animals (sham) (FIG. 5C(ii)).

Although some metabolites such as pyruvate and citrate are not detected in tissue samples, due to their immediate transformation upon death (post-mortem effect), these experiments show dysregulations of alanine and lactate levels common to serum and tissue samples.

Pramipexole Modifies Serum and Tissue Metabolic Dysregulations Induced by 6-OHDA

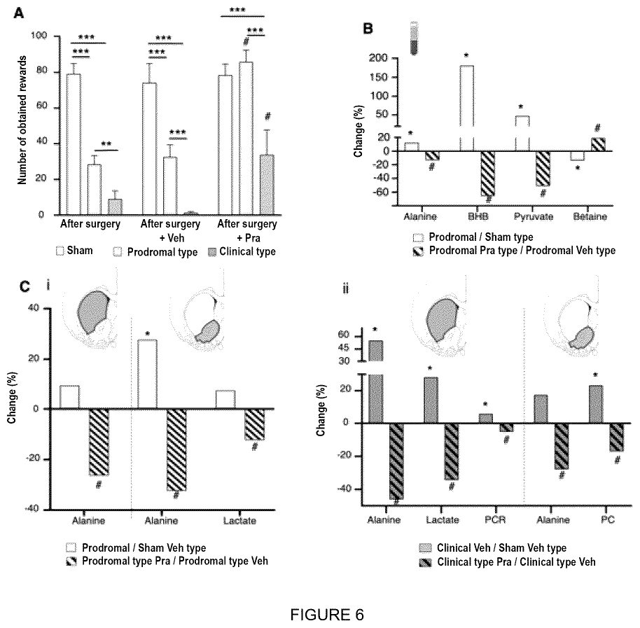

It was checked whether the serum metabolic profile was altered by a classically used clinical dopamine agonist, Pramipexole (Pra), known to improve only neuropsychiatric symptoms when used at low dose in animal models [23,24]. As expected, after 15 days of subchronic Pra administration, deficits in the self-administration task measured in 6-OHDA rats were fully or partially ameliorated in prodromal-type and clinical-type animals, respectively. On the other hand, the performance of Veh-treated rats was not improved (FIG. 6A).

With respect to serum metabolic profiles, to visualize simultaneously the effect of 6-OHDA lesion and treatment, metabolite levels of prodromal-like animals were normalized to the level of control (sham) animals (lesion effect, solid bar) and metabolite levels of Pra-treated prodromal-type animals were normalized to Veh-treated prodromal-type animals (Pra effect, hatched bar). In prodromal animals, alanine BHB and pyruvate levels increased after injury (FIGS. 4D and 6B, solid bar) and were significantly reduced by Pra (FIG. 6B, hatched bar), while betaine decreased after injury, but increased in Pra animals. In the clinical type group, only the alanine level, increased after the lesion, was slightly reduced by Pra, in accordance with the modest behavioral effect observed in self-administration and no effect on motor tasks, given the low dose of Pra [24] (data not shown). The other metabolites were deregulated in the same way in both groups, regardless of the received treatment (Veh or Pra).

Unlike serum, for which multiple longitudinal samples could be collected from the same animal, brain samples could only be collected at the end of the experiment, when all animals had received a Pra or Veh chronic treatment. Therefore, control (sham) animals treated with Veh were used as a reference to normalize metabolite levels for prodromal type animals treated with Veh (Lesion effect, solid bar). The metabolite levels of Pra-treated prodromal-type animals were normalized relative to prodromal-type animals given Veh (Pra effect, hatched bar). As in serum, administration of Pra caused a decrease in some metabolites previously increased by 6-OHDA lesion. That was true for alanine in DS and Nacc, and for lactate in Nacc in prodromal type animals (FIG. 6C i). In the clinical type group, alanine level after Pra was also reversed just as in prodromal type animals, and lactate was significantly altered in DS. In addition, reversion of phosphocreatine and phosphocholine levels was observed in DS and Nacc, respectively (FIG. 6C ii).

Overall, significant alterations in serum and tissue metabolites were measured in the 6-OHDA rat model and associated with the different phases of the disease and, interestingly, were detected as early as the prodromal stage. Moreover, some of these disturbances were partially reversed by chronic administration of Pra, which also corrected the neuropsychiatric deficits.

In order to test the specificity of the biomarkers found in the 6-OHDA model regarding disease progression and pathophysiological mechanisms, the serum metabolomics study was extended to two other animal models of PD.

6-OHDA, α-Synuclein Rats and MPTP Monkeys Share Serum Metabolic Disturbances

First, using the same strain of rat (Sprague Dawley) as for the 6-OHDA study, an α-synuclein rat model was selected, in which overexpression of human A53T alpha-synuclein was induced in SNc using adeno-associated viral (AAV) vectors. This targeted expression leads to progressive neurodegeneration in the targeted region, which makes it possible to follow the different stages of PD in the same animal [19], namely the prodromal-type phase around 3 weeks and the clinical-type phase around 10 weeks after the injection.

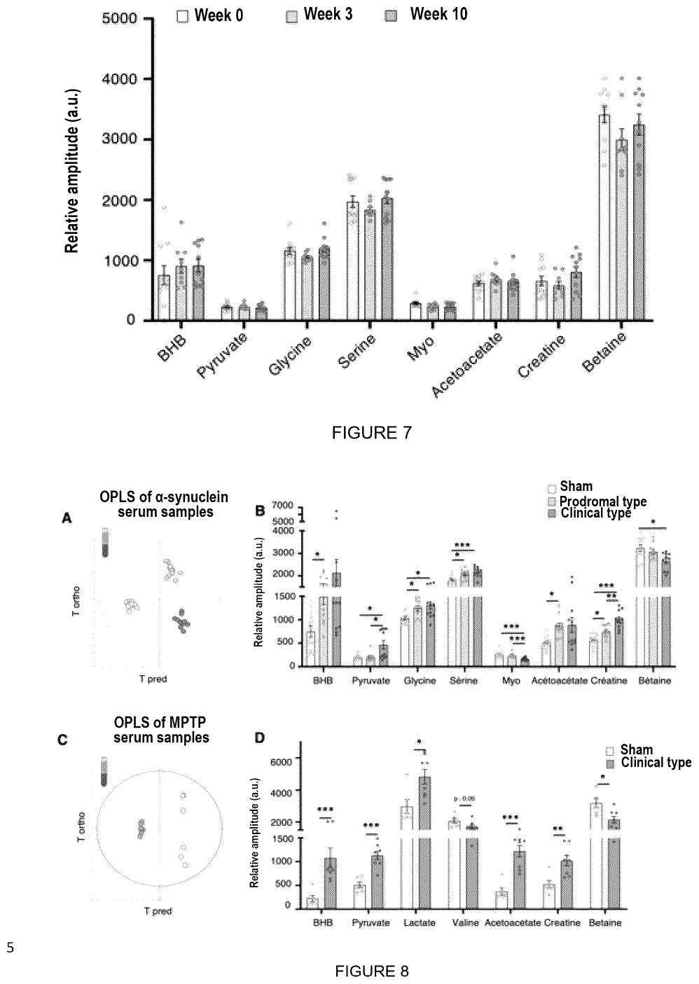

An OPLS-DA model was used to assess whether a specific metabolic signature was associated with these different phases (i.e. the «serum α-synuclein OPLS-DA model»). Sham animals (control injected with a comparison group virus encoding GFP) showed no difference between the three different time points 0, 3 and 16 weeks after injection (FIG. 7), while for α-synuclein animals, the 3 groups corresponding to the three different samples characteristic of the different phases are clearly separated in the OPLSD-DA model (FIG. 8A), indicating that each phase was characterized by a different metabolic signature. In particular, a significant increase in BHB, glycine, pyruvate, serine and a decrease in betaine were observed over time, as in 6-OHDA rats. Additionally, a significant decrease of myo-inositol and increase of acetoacetate and creatine were observed only in the α-synuclein model (FIG. 8B).

These results revealed similar metabolic disturbances between the two rat models of PD, which are associated with the disease progression. To go further, a metabolic analysis was carried out in a non-human primate model of PD, e.g. the MPTP model, which mimics the clinical phase of PD and shows greater homology with humans [25].

The OPLS-DA model shown in FIG. 8C (that is to say the «OPLSDA serum MPTP model») showed clear discrimination between control (sham) and MPTP groups. On the one hand, non-human MPTP primates exhibited a significant increase in lactate and decrease in valine, similar to 6-OHDA rats. On the other hand, they exhibited a significant increase in acetoacetate and creatine as in the α-synuclein rodent model. Finally, a significant increase in BHB and pyruvate was observed, as well as a significant decrease in betaine, as in the two rodent models (FIG. 8D).

Taken together, these results reveal common metabolic alterations associated with PD progression in three different animal models of PD, performed in two different species. Thus, to validate the clinical relevance of these markers, these preclinical results were compared with those obtained in samples from patients with PD.

Serum Metabolic Signatures of Newly Diagnosed Parkinson's Patients and Animal Models of PD Show Similarities

Each cohort was first analyzed individually, ie. newly diagnosed parkinsonian patients (n=21 in each cohort) compared to age- and sex-matched comparison groups (n=30/23 NIH/Italy).

With the exception of some metabolites, e.g. alanine (data not shown), the variations observed were consistent between the 2 cohorts, as shown in Table 3. The latter presents the main individual and collective changes in metabolites between patients with PD and controls in the Italian and United States (NIH) cohorts, analyzed individually by t-test and collectively by two-way ANOVA, followed by a Sidak post hoc test. *: p≤0.05; **: p≤0.01; ***: p≤0.001; ns: not significant.

TABLE 3

| | Italy | | NIH | | 2 ways Anova | | |
|---|---|---|---|---|---|---|---|
| | Change | p | Change | p | Cohorts | | inter- |
| | (%) | value | (%) | value | Origin | Case | action |
| Acetoacetate | 17.26 | 0.05 | 28.12 | 0.12 | ** | ** | ns |
| Betaine | −9.74 | 0.35 | −26.27 | 0.01 | ns | ** | ns |
| BHB | 28.28 | 0.24 | 103.23 | 0.09 | *** | * | ns |
| Leucine | 5.26 | 0.37 | 11.14 | 0.14 | *** | 0.08 | ns |
| Valine | −11.04 | 0.13 | −14.61 | 0.04 | *** | * | ns |

For example, a marginal increase in BHB and acetoacetate was observed in Italian PD patients (+28.3%, p=0.24 and +17.2%, p=0.05 respectively), and was even higher in patients from the United States (+103.2%, p=0.09 and +28.1%, p=0.12).

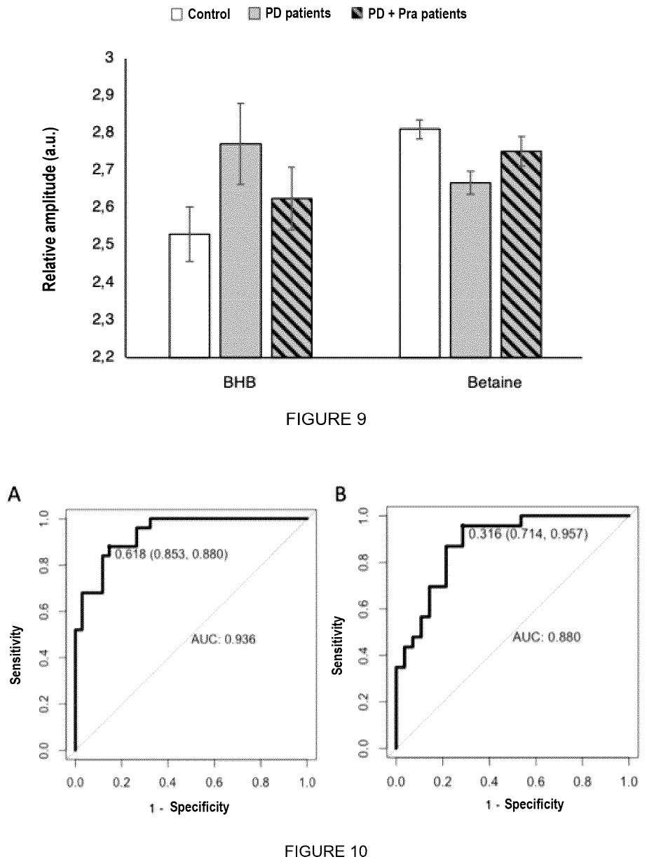

In order to increase the statistical power, the two cohorts were pooled (PD n=42, comparison group n=53). Nevertheless, since the variance of the data was mainly affected by the origin of the sample, that is to say Italy or the United States, independently of the case (PD or comparison group), a two-way ANOVA was performed with i) cohort origin and ii) case (comparison group or PD) as factors to extract PD-only information. Table 3 illustrates the significant metabolites for the case factor, with no interaction between the origin cohort and the case. The increase in BHB and acetoacetate, observed in each cohort, appeared significant when pooled, A significant decrease in betaine in the serum of patients with PD was also detected, as in all animal models. In addition, a significant decrease in valine was observed as in the 6-OHDA rat model. Finally, a decrease in leucine was observed. Additionally, the analysis of a small number of serum samples from other newly diagnosed and treated with Pra 0.5-100 mg/day (n=9) revealed a trend towards normalization of BHB and betaine levels compared to untreated PD patients, as observed in rats 6-OHDA (FIG. 9).

Serum Metabolites as Potential Markers for the Early Diagnosis of Parkinson's Disease

To assess the potential of the identified metabolites to distinguish prodromal type animals from control (sham) animals, multiple logistic regression models were developed, based on all possible combinations of levels of acetoacetate, betaine, creatine, BHB, pyruvate and valine, grouping prodromal-type 6-OHDA and α-synuclein rats.

The best regression model, evaluated by the ROC curves (see materials and methods), retained the six metabolites, using a regression based on the following predictive algorithm (p: 1.107×10⁻¹⁵):

Logit(P)=log(P/(1−P))=−54.72+0.49BHB+0.39Pyruvate−0.42Valine+0.06Acetoacetate+0.52Creatine−0.10Betaine

The ROC curve had an area under the curve (AUC) of 0.936. By applying the optimal threshold of 0.618 (sensitivity 0.85, specificity 0.88) (FIG. 10A), an accuracy of 0.925 was found, making it possible to correctly predict 37 animals among the 40 included in the validation cohort (rats clinical type and all primates).

Regarding human samples, the United States (NIH) cohort was used to build a model because i) it is the largest cohort and ii) it includes only recently diagnosed patients, unlike the Italian cohort, which includes patients with longer PD duration. The latter was used as an external validation cohort. Here again, the best model retained the six metabolites combined with the following regression algorithm (p: 1.95×10⁻⁴): Logit(P₁)=log(P₁/(1−P₁))=−45.60+0.28BHB−4.54Pyruvate−11.23Valine+7.05Acetoacetate+2.85Creatine−8.27Betaine.

The following ROC curve had an AUC of 0.88. The optimal threshold was 0.316, and the corresponding sensitivity and specificity were 0.957 and 0.714, respectively (FIG. 10B). The classification prediction of the Italian cohort detected 82.6% of patients with PD, with 17.4% false negatives.

The United States (NIH) cohort minus 2 unretained patients was also used to build a model. Here again, the best model retained the six metabolites combined with the following regression algorithm (p: 1.95×10⁻⁴):

Logit(P₂)=log(P₂/(1−P₂))=−42.78+0.16BHB−3.31Pyruvate−12.01Valine+7.06Acetoacetate+3.14Creatine−7.79Betaine.

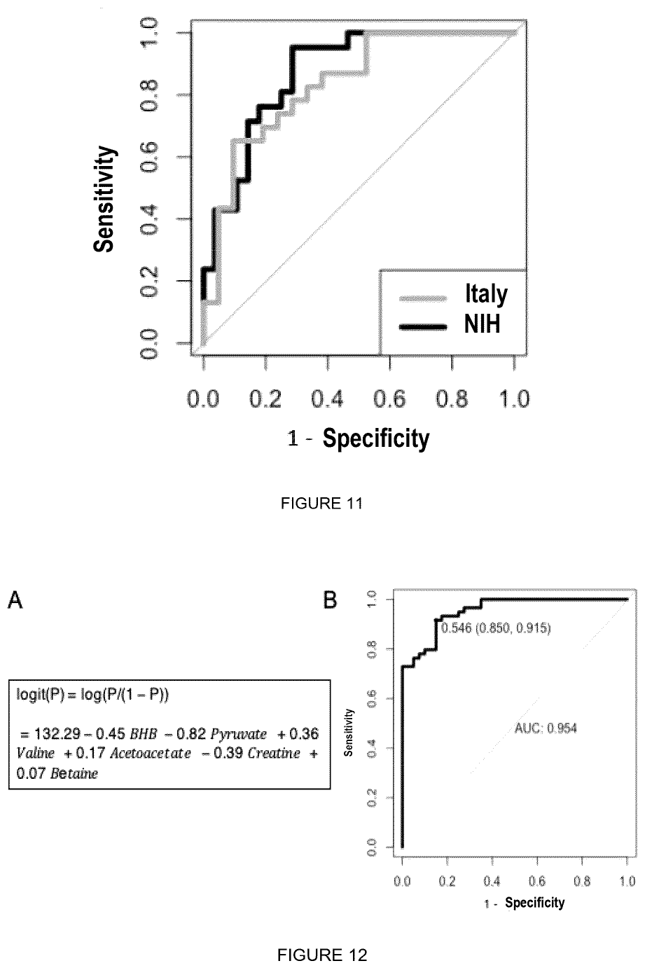

The following ROC curve had an AUC of 0.87. The optimal threshold was and the corresponding sensitivity and specificity were 0.952 and 0.714, respectively (FIG. 11, black line). The prediction of the classification of the Italian cohort allowed to detect 82.6% of patients with PD, with 17.4% false negatives. The ROC curve constructed with the Logit(P₂) regression algorithm and the Italian cohort had an AUC of 0.83. (FIG. 11, gray line).

Finally, multiple logistic regression was performed using serum data from all MP animals (prodromal and clinical type) from all models using the same six metabolites and was evaluated with the following ROC curve showing an AUC of (FIG. 12) and an accuracy of 88.9%.

In total, the precision of the biomarker developed from the same six serum metabolites was at least 82.6% regardless of the species.

### LIST OF REFERENCES

1. Samii A, Nutt J G, Ransom B R. Parkinson's disease. Lancet, 2004; 363: 1783-1793.doi: 10.1016/S0140-6736(04)16305-8
2. Postuma R B, Berg D, Stern M, et al. MDS clinical diagnostic criteria for Parkinson's disease. Mov Disord. 2015. doi:10.1002/mds.26424
3. Hughes A J, Ben-Shlomo Y, Daniel S E, Lees A J. What features improve the accuracy of clinical diagnosis in Parkinson's disease: A clinicopathologic study.
4. Stocchi F. Therapy for Parkinson's Disease: What is in the Pipeline? Neurotherapeutics. 2013; 11:24-33. doi: 10.1007/s13311-013-0242-1
5. Adler C H, Beach T G, Hentz J G, et al. Low clinical diagnostic accuracy of early vs advanced Parkinson disease: Clinicopathologic study. Neurology. 2014. doi:10.1212/WNL.0000000000000641
6. Hughes A J, Daniel S E, Kilford L, Lees A J. Accuracy of clinical diagnosis of idiopathic Parkinson's disease: A clinico-pathological study of 100 cases. J Neurol Neurosurg Psychiatry. 1992. doi:10.1136/jnnp.55.3.181
7. Hughes A J, Ben-Shlomo Y, Daniel S E, Lees A J. What features improve the accuracy of clinical diagnosis in parkinson's disease: A clinicopathologic study. Neurology. 2001. doi:10.1212/wn1.42.6.1142
8. Salamon A, Zädori D, Szpisjak L, Klivényi P, Vécsei L. Neuroprotection in Parkinson's disease: facts and hopes. J Neural Transm. 2020.doi:10.1007/s00702-019-02115-8
9. Olanow C W, Kieburtz K, Schapira A H V. Why have we failed to achieve neuroprotection in Parkinson's disease? Ann Neurol. 2008; 64(Supp. 2). doi:10.1002/ana.21461
10. Beal M F, Rascal, Marek, et al. Bioenergetic approaches for neuroprotection in Parkinson's disease. Ann Neurol. 2003. doi:10.1002/ana.10479
11. Jankovic J. Parkinson's disease: Clinical features and diagnosis. J Neurol Neurosurg Psychiatry. 2008. doi: 10, 1136/jnnp.2007.131045
12. Michell A W, Lewis S J G, Foltynie T, Barker R A. Biomarkers and Parkinson's illness. Brain 2004. doi:10.1093/brain/awh198
13. Chaudhuri K R, Healy D G, Schapira a H. Non-motor symptoms of Parkinson's disease: diagnosis and management. Lancet Neural. 2006; 5(3):235-245. doi:10.1016/s1474-4422(06)70373-8
14. Kaur R, Mehan S, Singh S. Understanding multifactorial architecture of Parkinson's disease: pathophysiology to management. Neural Sci. 2019. doi:10.1007/s10072-018-3585-x
15. Johnson C H, González FJ. Challenges and opportunities of metabolomics. J Cell Physical. 2012. doi:10.1002/jcp.24002
16. Ala-Korpela M. Potential role of body fluid 1H NMR metabonomics as a prognostic and diagnostic tool. Expert Rev Mol Diagn. 2007; 7(6):761-773. doi:10.1586/147371591.6.761
17. Hatano T, Saiki S, Okuzumi A, Mohney R P, Hattori N. Identification of novel biomarkers for parkinson's disease by metabolomic technologies. J Neurol Neurosurg Psychiatry. 2015:1-7. doi:10.1136/jnnp-2014-309676
18. Bogdanov M, Matson W R, Wang L, et al. Metabolomic profiling to develop blood biomarkers for Parkinson's disease. Brain. 2008; 131(2):389-396, doi: 10.1093/brain/awm 304
19. Lei S, Powers R. NMR Metabolomics Analysis of Parkinson's Disease. Curr Metabolomics, 2013; 1(3): 191-209, doi:10.2174/2213235X113019990004
20. Quinones M P, Kaddurah-Daouk R. Metabolomics tools for identifying biomarkers for neuropsychiatric diseases, Neurobiol Dis. 2009; 35(2):165-176. doi:10.1016/j.nbd.2009.02.019
21. Nagesh Babu G, Gupta M, Paliwal V K, Singh S, Chatterji T, Roy R. Serum metabolomics study in a group of Parkinson's disease patients from northern India. Clin Chim Acta. 2018; 480(January):214-219. doi:10.1016/j.cca.2018.02.022
22. Lewitt P A, Li J, Lu M, Guo L, Auinger P. Metabolomic biomarkers as strong correlates of Parkinson disease progression. Neurology. 2017; 88(9):862-869, doi:10.1212/WNL.0000000000003663
23. Vingill S, Connor-Robson N, Wade-Martins R. Are rodent models of Parkinson's disease behaving as they should? Behav Brain Res. 2018. doi:10.1016 bbr.2017.10.021
24. Drui G, Carnicella S, Carcenac C, et al. Loss of dopaminergic nigrostriatal neurons accounts for the motivational and affective deficits in Parkinson's disease. Mol Psychiatry. 2014; 19(3): 358-367. doi: 10.1038/mp.2013.3
25. Bourdenx M, Dovero S, Engeln M, et al. Lack of additive role of aging in nigrostriatal neurodegeneration triggered by α-synuclein overexpression. Acta Neuropathol Commun. 2015. doi:10.1186/s40478-015-0222-2
26. Seo J, Lee Y, Kim B S, et al. A non-human primate model for stable chronic Parkinson's disease induced by MPTP administration based on individual behavior quantification. J Neurosci Methods. 2019. doi:10.1016/j.jneumeth.2018.10.037
27. Wong M L, Licinio J. Peripheral is Central to the question. Mol Psychiatry. 2005. doi: 10.1038/4 mp.4001674
28. Magnard R, Vachez Y, Carcenac C, et al. What can rodent models tell us about apathy and associated neuropsychiatric symptoms in Parkinson's disease? Transl Psychiatry. 2016:6(3);e753. doi:10.1038/tp.2016.17
29. Decressac M, Mattsson B, Lindblad M, Weikop P, Bjorklund A. Progressive neurodegenerative and behavioral changes induced by AAV-mediated overexpression of α-synuclein in midbrain dopamine neurons. Neurobiol Dis. 2012. doi:10.1016/jnbd.2011.12.013
30. Dehay B, Fernagut P O. Alpha-synuclein-based models of Parkinson's disease. Rev Neurol (Paris). 2016. doi:10.1016/j.neurol.2016.04.003
31. Teahan O, Gamble S, Holmes E, et al. Impact of analytical bias in metabonomics studies of human blood serum and plasma. Anal Chem. 2006. doi:10.1021/ac051972y
32. Lôpez-Bascôn M A, Priego-Capote F, Peralbo-Molina A, Calderôn-Santiago M, Luque De Castro M D. Influence of the collection tube on metabolomic changes in serum and plasma. Talanta. 2016. doi:10.1016/j.talanta.2015.12.079
33. Pagano G, Niccolini F, Politis M. Imaging in Parkinson's disease. Clin Med JR Coil Physicians London. 2016. doi:10.7861/clinmedicine.16-4-371

1. An in vitro method for early diagnosis of a neurodegenerative disease from a biological sample of a patient comprising:
a) the in vitro measurement in the biological sample of the expression levels of at least the following 6 metabolites: acetoacetate, betaine, β-hydroxybutyrate (BHB), creatine, pyruvate, and valine;
b) comparison of the expression levels of the metabolites measured in step a) with those of a reference biological sample or with reference values;
where a difference in the expression levels of the metabolites measured in step a) compared to those of the reference biological sample or to reference values is the indication that the patient is suffering from the neurodegenerative disease.

2. The method according to claim 1, wherein step a) further comprises the in vitro measurement of the expression level of at least one metabolite selected from the group consisting of alanine, lactate, DMSO2, glycine, serine, threonine, myoinositol, and leucine.

3. The method according to claim 1, wherein step a) comprises the measurement of the expression levels of the following metabolites: acetoacetate, betaine, β-hydroxybutyrate (BHB), creatine, pyruvate, and valine.

4. The method according to claim 1, wherein the neurodegenerative disease is Parkinson's disease.

5. The in vitro method for the early diagnosis of Parkinson's disease from a biological sample from a patient comprising:
a) the in vitro measurement in the biological sample of the expression levels of the following metabolites: acetoacetate, betaine, β-hydroxybutyrate (BHB), creatine, pyruvate, and valine;
b) calculation of Logit(P1) or Logit(P2) logistic regressions, respectively, according to the following formulas:
−45.60+0.28EBHB−4.54EPyruvate−11.23EValine+7.05EAcetoacetate+2.85ECreatine−7.79EBetaine; or
−42.78+0.16EBHB−3.31EPyruvate−12.01EValine+7.06EAcetoacetate+3.14ECreatine−8.27EBetaine,
where EBHB, EPyruvate, EValine, EAcetoacetate, ECreatine and EBetaine correspond respectively to the measured expression levels of β-hydroxybutyrate (BHB), pyruvate, valine, acetoacetate, creatine and betaine, in the biological sample;
and
where a calculated value of Logit(P1) or Logit(P2) greater than 0.316 a.u. is an indication that the patient has Parkinson's disease;
where a calculated value of Logit(P1) or Logit(P2) lower than 0.279 a.u. is an indication that the patient does not have Parkinson's disease;
where a calculated value of Logit(P1) less than 0.316 a.u. is an indication that the patient does not have Parkinson's disease;
where a calculated value of Logit(P2) greater than 0.279 a.u. is an indication that the patient has Parkinson's disease.

6. The method according to claim 4, wherein the patient's biological sample comes from a patient in the prodromal or de novo stage of Parkinson's disease.

7. The method according to claim 1, wherein the biological sample is a serum sample.

8. A biomarker for early diagnosis of a neurodegenerative disease comprising the combination of at least six of the following metabolites: acetoacetate, betaine, β-hydroxybutyrate (BHB), creatine, pyruvate, and valine.

9. The biomarker according to claim 7 further comprising the combination of at least one metabolite selected from the group consisting of alanine, lactate, DMSO2, glycine, serine, threonine, myo-inositol, and leucine.

10. The biomarker according to claim 8, comprising the combination of the following six metabolites: acetoacetate, betaine, β-hydroxybutyrate (BHB), creatine, pyruvate, and valine.

11. An in vitro use of a biomarker according to claim 8, as a biomarker of a neurodegenerative disease.

12. The use according to claim 11, wherein the neurodegenerative disease is Parkinson's disease.
